(19) United States
(12) Patent Application Publication
(10) Pub. No.: US 2020/0030206 A1
(43) Pub. Date: Jan. 30, 2020

(54) NAIL POLISH COMPOSITION SYSTEM AND METHOD OF APPLYING SAME

(71) Applicant: Chin Hao Chang, New Taipei City (TW)

(72) Inventor: Chin Hao Chang, New Taipei City (TW)

(21) Appl. No.: 16/521,417

(22) Filed: Jul. 24, 2019

Related U.S. Application Data
(60) Provisional application No. 62/703,829, filed on Jul. 26, 2018.

(51) Int. Cl. A61K 8/41; A61K 8/81; A61K 8/37; A61Q 3/02

(52) U.S. Cl. CPC A61K 8/411; A61K 8/8152; A61K 2800/594; A61Q 3/02; A61K 8/37

(57) ABSTRACT

A nail polish composition system includes (a) a base coat nail polish composition including organic solvents, film-forming compounds, plasticizers, and one or more additives; and (b) a top coat nail polish composition including a mixture of cyanoacrylates and a methacrylate. The base coat nail polish composition is applied to a fingernail to form a base coat film. Thereafter, the top coat nail polish composition is applied on or over the base coat film to form a top coat film. The nail polish composition system can significantly shorten the time required for drying the base and top coat nail polish compositions. In addition, the nail polish composition system may be applied without dipping the fingernail in powder glaze.

### CROSS REFERENCE TO RELATED APPLICATIONS

The present application claims priority to U.S. Provisional Patent Application Ser. No. 62/703,829, filed, Jul. 26, 2018, the disclosure of which is incorporated by reference herein in its entirety.

### FIELD OF THE INVENTION

The invention relates to a nail polish composition system and method, and more particularly, to a fast drying system of nail polish compositions and a method of applying same.

### BACKGROUND OF INVENTION

Consumers use nail art to paint, decorate, and enhance their nails cosmetically. Typically, the process of applying nail polish compositions includes application of several layers of coatings, each of which is kept to dry. For example, one may first apply one or two layers of base coat, followed by application of one or two layers of top coat to provide glossiness and protection.

Commercial nail polish compositions normally comprise organic solvents, film-forming compounds, and plasticizers. Each base coat and top coat can take more than several minutes to dry. While consumers wait for the base and top coats to dry, they may be concerned that the surface of the nail polish may become defective due to, for instance, collision. Due to these concerns, consumers tend to remain stationary until the top and base coats are completely dry, thereby experiencing inconvenience. In addition, fingernails may be dipped in powder glaze, potentially creating a troublesome working environment.

### SUMMARY OF THE INVENTION

It has been discovered that applying a base coat nail polish composition including dimethyltolylamine (N, N-dimethyl-p-toluidine), followed by application of a top coat nail polish composition including a mixture of cyanoacrylates and a methacrylate can significantly shorten the drying time.

In one embodiment, a nail polish composition system includes a base coat nail polish composition including dimethyltolylamine and a top coat nail polish composition including a mixture of cyanoacrylates and a methacrylate. The base coat nail polish composition is applied on a fingernail one or more times (e.g., one, two or three times) to to form a base coat film. The top coat nail polish composition is subsequently applied one or more times (e.g., one, two or three times) on or over the base coat film to form a top coat film.

In one embodiment, a nail polish composition system includes a base coat nail polish composition including dimethyltolylamine and a top coat nail polish composition including at least one cyanoacrylate. In one embodiment, the top coat nail polish further includes at least another cyanoacrylate and/or methacrylate. The base coat nail polish composition is applied on a fingernail one or more times (e.g., one, two or three times) to form a base coat film. The top coat nail polish composition is subsequently applied one or more times (e.g., one, two or three times) on or over the base coat film to form a top coat film.

In one embodiment, a nail polish composition system includes a base coat nail polish composition and a top coat nail polish composition including at least one cyanoacrylate. In one embodiment, the bottom coat nail polish composition includes dimethyltolylamine, while the top coat nail polish includes at least another cyanoacrylate and/or methacrylate. The base coat nail polish composition is applied on a fingernail one or more times (e.g., one, two or three times) to form a base coat film. The top coat nail polish composition is subsequently applied one or more times (e.g., one, two or three times) on or over the base coat film to form a top coat film.

In another embodiment, the invention relates to a nail polish composition system including: (a) a base coat nail polish composition including organic solvents, film-forming compounds, plasticizers, and one or more additives; and (b) a top coat nail polish composition including a mixture of cyanoacrylates and a methacrylate. In one embodiment, the one or more additives may include dimethyltolylamine. The base coat nail polish composition is applied on a fingernail one or more times (e.g., one, two or three times) to form a base coat film. The top coat nail polish composition is subsequently applied one or more times (e.g., one, two or three times) on or over the base coat film to form a top coat film.

In a further embodiment, besides organic solvents, film-forming compound, plasticizer, and one or more additives, the base coat nail polish composition further includes additional optional components to enhance performance.

In one embodiment, the base coat nail polish composition contains the following ranges: organic solvents are present in the range of from about 40 to about 60 wt % of the base coat nail polish composition; film-forming compounds are present in the range of from about 20 to about 30 wt % of the base coat nail polish composition; plasticizers are present in the range of from about 5 to about 15 wt % of the base coat nail polish composition; one or more additives are present in the range of from about 0.01 to about 5 wt % of the base coat nail polish composition; and additional components are present in the range of from about 5 to about 10 wt % of the base coat nail polish composition.

The top coat nail polish composition preferably contains the following ranges: Beta-methoxyethyl cyanoacrylate is present in the range of from about 70 to about 95 wt % of the mixture of cyanoacrylates and a methacrylate; C1-C10 linear or branched alkyl cyanoacrylate is present in the range of from about 5 to about 20 wt % of the mixture of cyanoacrylates and a methacrylate; and polymethyl methacrylate is present in the range of from about 1 to about 10 wt % of the mixture of cyanoacrylates and a methacrylate.

In one embodiment, application of a base coat nail polish composition including organic solvents, film-forming compounds, plasticizers and one or more additives eliminates dipping fingernails in powder glaze.

### DETAILED DESCRIPTION OF THE INVENTION

It should be noted that terms, such as “first”, “second”, “one”, “a”, “an”, and the like, are for illustrative purposes only, and are not meant to limit the range implementable by the invention. Any changes or adjustments made to their relative relationships, without modifying the substantial technical contents, are also to be construed as within the range implementable by the invention.

As used herein, the term “nail polish composition” is intended as a broad term covering a nail polish layer, film, product, system, kit, or the like, which is useful for providing aesthetic or therapeutic benefits to the nail.

As used herein, a “base coat” is a layer of nail polish composition. The base coat is preferably applied contiguously to nail with one or more additional layers applied to the resulting base coat.

As used herein, a “top coat” is a layer of nail polish composition. The top coat is preferably applied contiguously to one or two base coats.

The invention relates to base coat nail polish compositions that contain one or more organic solvents, one or more film-forming compounds, one or more plasticizers, and one or more additives.

In one embodiment, the organic solvents have a boiling point ranging from about 50° C. to about 150° C. at 1 atm. In one embodiment, the organic solvents are selected from C1-C10 alcohols, esters, ketones, aromatic hydrocarbons, aliphatic hydrocarbons, ethers, and/or mixtures thereof. In one embodiment, the alcohols are monohydric. In one embodiment, the monohydric alcohols may be ethanol, 1-propanol, 2-propanol, isopropanol (isopropyl alcohol), and/or n-butyl alcohol. In one embodiment, esters may be butyl, ethyl, isopropyl and/or propyl acetate. Other non-limiting examples of the organic solvents include benzyl alcohol, amyl acetate, acetone, heptane, toluene, methyl acetate, 1-butanol, 1-amyl alcohol, 2-butyl alcohol, hexane, and/or methyl ethyl ketone. In one embodiment, the organic solvents may be selected from isopropanol, n-propanol, ethanol, n-butyl alcohol, ethyl acetate, butyl acetate, and/or acetone.

In some embodiments, the organic solvents are present in the range of from about 30 to about 70 wt %, from about 40 to about 70 wt %, or from about 40 to about 60 wt %, of the base coat nail polish composition.

In one embodiment, the film-forming compounds are generally solvent-borne polymers, such as cellulosic polymers or acrylate copolymers. The film-forming polymers can be nonionic, ionic, and/or amphoteric. In one embodiment, the film-forming polymers are self-curing such that they may be cured without any chemical reaction or introduction of energy in order to form an adherent continuum on nails.

In one embodiment, the cellulosic polymers are nitrocellulose, cellulose esters, such as cellulose acetate, cellulose acetate butyrate, cellulose acetate propionate, and/or mixtures thereof.

Combination of nitrocellulose polymers with polyvinyl derivatives such as polyvinyl butyral is also possible.

Examples of the acrylate copolymers can be formed from monomers including unsaturated carboxylic acid such as acrylic acid, methacrylic acid, crotonic acid, fumaric acid, and/or itaconic acid; alkyl acrylates such as methyl acrylate, ethyl acrylate, propyl acrylate, butyl acrylate, pentyl acrylate, and/or hexyl acrylate; alkoxyalkyl acrylates such as methoxyethyl acrylate, ethoxyethyl acrylate, propoxyethyl acrylate, butoxyethyl acrylate and/or ethoxypropyl acrylate; alkyl methacrylates such as methyl methacrylate, ethyl methacrylate, propyl methacrylate, butyl methacrylate, pentyl methacrylate, and/or hexyl methacrylate; alkoxyalkyl methacrylates such as methoxyethyl methacrylate, ethoxyethyl methacrylate, propoxyethyl methacrylate, butoxyethyl methacrylate and/or ethoxypropyl methacrylate; (poly)alkylene glycol diacrylates such as ethylene glycol diacrylate, diethylene glycol diacrylate, triethylene glycol diacrylate, poly(ethylene glycol) diacrylate, propylene glycol diacrylate, dipropylene glycol diacrylate, and/or tripropylene glycol diacrylate; and/or polyvalent acrylates such as trimethylolpropane triacrylate. In one embodiment, butyl acrylate/methyl methacrylate/methacrylic acid copolymers or styrene/acrylates copolymers may be utilized. In another embodiment, adipic acid/neopentyl glycol/trimellitic anhydride copolymers can also be used as film-forming compounds.

In some embodiments, the film-forming compounds are present in the range of from about 10 to about 40 wt %, from about 20 to about 40 wt %, or from about 20 to about 30 wt %, of the base coat nail polish composition.

In one embodiment, the plasticizer can enter the interstitial sites of the film-forming compounds to modify the flexibility of the formed nitrocellulose/acrylate copolymer matrix, causing the composition to become more easily deformed. In one embodiment, the plasticizers may include those disclosed in WO 97/00664, Chen et al, assigned to Eastman Chemical Co., the entire disclosure of which is incorporated herein by reference.

In further embodiments, the plasticizers include phthalates, citrates, nonionic surfactant polymers, polyesters, and/or mixtures thereof. In one embodiment, the phthalates may include diethyl phthalate, dibutyl phthalate, and/or dioctyl phthalate, while the citrates may include acetyl tributyl citrate. In another embodiment, the nonionic surfactant polymers may include tartrates, (e.g., diethyl tartrate and/or dibutyl tartrate), phosphates (e.g., diethyl phosphate and/or dibutyl phosphate) and/or glycols (e.g., tetraethylene glycol di-2-ethylhexoate). Other plasticizers include camphor, sucrose acetate isobutyrate, trimethylpentanediyl dibenzoate and/or castor oil.

In some embodiments, the plasticizers are present in the range of from about 2 to about 30 wt %, from about 5 to about 20 wt %, or from about 5 to about 15 wt %, of the base coat nail polish composition.

In one embodiment, the base coat nail polish compositions of the present invention may comprise additional optional components to enhance their performance. For example, pigments, anti-foaming agents, thickening agents, suspension agents, UV absorbers, and/or the like can be added. Such optional components may be dispersed, solubilized, mixed, or added to the base coat nail polish compositions provided they do not substantially impair the fast drying property of the compositions.

In one embodiment, the pigments may be inorganic or organic pigments, such as those are disclosed in the C.T.F.A. Cosmetic Ingredient Handbook, First Edition, 1988. Organic pigments include D&C Red, No. 5, 6, 7, 10, 11, 12, 13, 30 and 34, lacquers such as D&C Yellow No. 5 and/or D&C Red No. 2, and/or guanine. Inorganic pigments include mica, titanium dioxide, bismuth oxychloride, and/or iron oxides.

In one embodiment, the anti-foaming agents may include acetylenic diol surfactants, trimethylsiloxysilicate, and/or dimethicone. In another embodiment, the thickening agents may include clays (e.g., stearalkonium bentonite), cellulose derivatives, silicates, and/or natural gums. In yet another embodiment, the suspension agents may include silica and/or alumina, while the UV absorbers may include ectocrylene, hydroxy benzotriazole compounds and/or benzophenone compounds.

The above mentioned components may have functions not limited to the listed category. For example, the anti-foaming agents may also function as leveling agents, while the thickening agents may also function as suspension agents. In addition, some film-forming compounds may function as plasticizers or adhesion promoters.

In some embodiments, the additional components are present in the range of from about 3 to about 15 wt %, from about 5 to about 15 wt %, or from about 5 to about 10 wt %, of the base coat nail polish composition.

In one embodiment, dimethyltolylamine (N, N-dimethyl-p-toluidine) is added to base coat nail polish compositions, which is applied to nails followed by application of a top coat nail polish composition comprising a mixture of cyanoacrylates and a methacrylate.

In some embodiments, the dimethyltolylamine are present in the range of from about 0.01 to about 10 wt %, from about 0.01 to about 8 wt %, or from about 0.01 to about 5 wt %, of the base coat nail polish composition.

The base coat nail polish composition can be made by mixing the components discussed above in any manner known to a person skilled in the art.

In one embodiment, a top coat nail polish composition includes a mixture of one or more cyanoacrylates and a methacrylate. In another embodiment, the top coat nail polish composition includes at least one cyanoacrylate and optionally a methacrylate. In yet another embodiment, the top coat nail polish composition includes cyanoacrylates and optionally methacrylate. It has been discovered that when used in conjunction with a base coat nail polish composition, which may contain dimethyltolylamine (N, N-dimethyl-p-toluidine), the top coat nail polish composition can significantly shorten the drying time. In one embodiment, the cyanoacrylates include Beta-methoxyethyl cyanoacrylate and/or C1-C10 linear or branched alkyl cyanoacrylate (or a combination thereof), while the methacrylate includes polymethyl methacrylate. In one embodiment, the top coat nail polish composition includes the following ranges: Beta-methoxyethyl cyanoacrylate is present in the range of from about 70 to about 95 wt % of the mixture of cyanoacrylates and a methacrylate; C1-C10 linear or branched alkyl cyanoacrylate is present in the range of from about 5 to about 20 wt % of the mixture of cyanoacrylates and a methacrylate; and polymethyl methacrylate is present in the range of from about 1 to about 10 wt % of the mixture of cyanoacrylates and a methacrylate. In one embodiment, the top coat nail polish composition may be formed by any manner known to a person skilled in the art.

### Examples

Non-limiting examples according to the invention are provided below, with a listing of the components and their wt % for each exemplary composition. One of ordinary skill in the art can readily understand the advantages and effects of the present invention upon reading the disclosure of this specification. The present invention may also be practiced or applied with other different implementations. Based on different contexts and applications, the various details in this specification can be modified and changed without departing from the spirit of the disclosure.

The nail polish composition of the invention can be made by combining its components, optionally with stirring. More generally, any known mixing method may be used.

Base Coat Nail Polish Composition 1

| Component | CAS # | wt % |
|---|---|---|
| Butyl Acetate | 123-86-4 | 28.93 |
| Ethyl Acetate | 141-78-6 | 20.50 |
| Nitrocellulose | 9004-70-0 | 17.00 |
| Adipic Acid/Neopentyl | | |
| Glycol/Trimellitic Anhydride | 28407-73-0 | 9.44 |
| Copolymer | | |
| Acetyl Tributyl Citrate | 77-90-7 | 8.00 |
| Isopropyl Alcohol | 67-63-0 | 2.00 |
| Acrylates Copolymer | 25035-69-2 | 1.50 |
| Dimethyltolylamine | 99-97-8 | 1.50 |
| Stearalkonium Bentonite | 130501-87-0 | 1.20 |
| N-Butyl Alcohol | 71-36-3 | 1.10 |
| Styrene/Acrylates Copolymer | 9010-92-8 | 1.10 |
| Silica | 7631-86-9 | 1.00 |
| Benzophenone-1 | 131-56-6 | 0.80 |
| Trimethylpentanediyl Dibenzoate | 68052-23-3 | 0.80 |
| Polyvinyl Butyral | 63148-65-2 | 0.05 |
| Alumina | 1344-28-1 | 0.03 |
| Dimethicone | 63148-62-9 | 0.03 |
| Trimethylsiloxysilicate | 56275-01-5 | 0.01 |
| Mica | 12001-26-2 | 3.50 |
| Titanium Dioxide | 13463-67-7 | 0.80 |
| D&C Red 7 | 5281-04-9 | 0.30 |
| Iron Oxide | 1309-37-1 | 0.20 |
| D&C Red 6 | 5858-81-1 | 0.20 |
| TOTAL | | 100.00 |

Top Coat Nail Polish Composition 2

| Component | CAS # | wt % |
|---|---|---|
| Beta-Methoxyethyl Cyanoacrylate | 027816-23-5 | 85.00 |
| 2-Octyl Cyanoacrylate | 133978-15-1 | 10.00 |
| Polymethyl Methacrylate | 9011-14-7 | 5.00 |
| TOTAL | | 100.00 |

Base Coat Nail Polish Composition 3 (Comparative)

| Component | CAS # | wt % |
|---|---|---|
| Butyl Acetate | 123-86-4 | 27.93 |
| Ethyl Acetate | 141-78-6 | 23.00 |
| Nitrocellulose | 9004-70-0 | 17.00 |
| Adipic Acid/Neopentyl | | |
| Glycol/Trimellitic Anhydride | 28407-73-0 | 9.44 |
| Copolymer | | |
| Acetyl Tributyl Citrate | 77-90-7 | 8.00 |
| Isopropyl Alcohol | 67-63-0 | 2.00 |
| Acrylates Copolymer | 25035-69-2 | 1.50 |
| Stearalkonium Bentonite | 130501-87-0 | 1.20 |
| N-Butyl Alcohol | 71-36-3 | 1.10 |
| Styrene/Acrylates Copolymer | 9010-92-8 | 1.10 |
| Silica | 7631-86-9 | 1.00 |
| Benzophenone-1 | 131-56-6 | 0.80 |
| Trimethylpentanediyl Dibenzoate | 68052-23-3 | 0.80 |
| Polyvinyl Butyral | 63148-65-2 | 0.05 |
| Alumina | 1344-28-1 | 0.03 |
| Dimethicone | 63148-62-9 | 0.03 |
| Trimethylsiloxysilicate | 56275-01-5 | 0.01 |
| Mica | 12001-26-2 | 3.50 |
| Titanium Dioxide | 13463-67-7 | 0.80 |
| D&C Red 7 | 5281-04-9 | 0.30 |
| Iron Oxide | 1309-37-1 | 0.20 |
| D&C Red 6 | 5858-81-1 | 0.20 |
| TOTAL | | 100.00 |

Top Coat Nail Polish Composition 4 (Comparative)

| Component | CAS # | wt % |
|---|---|---|
| Ethyl Acetate | 141-78-6 | 32.91 |
| N-Butyl Acetate | 123-86-4 | 29.00 |
| Nitrocellulose | 9004-70-0 | 15.00 |
| Phthalic Alkyd Resin | 63148-69-6 | 12.59 |
| Isopropyl Alcohol | 67-63-0 | 10.00 |
| Butyl Methoxydibenzoylmethane | 70356-09-1 | 0.50 |
| TOTAL | | 100.00 |

### Drying Tests

Put a commercially available scraper with 60 mm thickness on top of a commercially available PET plastic sheet with a 20×20 cm size and drop 5 g of Base Coat Nail Polish Composition 1 and Base Coat Nail Polish Composition 3 in front of the 60 mm thick scraper. Scratch the 60 mm thick scraper to produce first base coat nail polish layers with 60 mm thickness. Wait for three minutes, and put a scraper with 100 mm thickness in front of the first base coat nail polish layers. Drop another 5 g of Base Coat Nail Polish Composition 1 and Base Coat Nail Polish Composition 3 in front of the 100 mm thick scraper, and scratch the 100 mm thick scraper to produce second base coat nail polish layers with 100 mm thickness on the first base coat nail polish layers. Wait for three minutes and put a scraper with 200 mm thickness in front of the second base coat nail polish layers. Drop 10 g of Top Coat Nail Polish Composition 2 and Top Coat Nail Polish Composition 4 in front of the 200 mm thick scraper, and scratch the 200 mm thick scraper to form top coat nail polish layers uniformly on the second base coat nail polish layers. Wait for five minute, spread 10 g of commercially available edible salts on first ⅓ regions of the top coat nail polish layers and immediately turn it upside down to check if the edible salts adhere to the top coat nail polish layers. Wait for another five minutes, spread another 10 g of edible salts on second untested ⅓ regions of the top coat nail polish layers and immediately turn it upside down to check if the edible salts adhere to the top coat nail polish layers. Finally, wait for another ten minutes, spread another 10 g of edible salts on the final untested ⅓ regions of the top coat nail polish layers and immediately turn it upside down to check if the edible salts adhere to the top coat nail polish layers. The drying results of the nail polish composition systems are shown in Table 1.

TABLE 1

| | 5 minutes | 10 minutes | 20 minutes |
|---|---|---|---|
| Example 1 | No salts adhesion | No salts adhesion | No salts adhesion |
| Comparative | Salts adhesion | Salts adhesion | Slight salts adhesion |
| Example 1 | | | |

The results shown in Table 1 confirm that the nail polish composition system according to the present disclosure can significantly reduce the drying time of nail polish composition.

In one embodiment, when applying a nail polish composition system to a fingernail, comprising the steps of: (a) applying a base coat nail polish composition, comprising organic solvents, film-forming compounds, plasticizers, and one or more additives, to the surface of the finger nail to form a first base coat film; (b) applying a top coat nail polish composition, comprising a mixture of cyanoacrylates and a methacrylate, over the first base coat film to form a first top coat film. The method can avoid dipping the fingernail in powder glaze by replacing the powder glaze with the base coat nail polish composition. In one embodiment, step (a) and step (b) can be repeated once. In another embodiment, step (a) and step (b) can be repeated twice.

It should be understood that the embodiments described herein are merely exemplary and that a person skilled in the art may make many variations and modifications without departing from the spirit and scope of the invention. All such variations and modifications are intended to be included within the scope of the invention and the appended claims.

I claim:

1. A nail polish composition system comprising:
(a) a base coat nail polish composition including organic solvents, film-forming compounds, plasticizers, and one or more additives; and
(b) a top coat nail polish composition including a mixture of cyanoacrylates and a methacrylate.

2. The nail polish composition system of claim 1, wherein the base coat nail polish composition includes additional components selected from a group consisting of pigments, anti-foaming agents, thickening agents, suspension agents, UV absorbers and/or a combination thereof.

3. The nail polish composition system of claim 1, wherein the one or more additives include dimethyltolylamine.

4. The nail polish composition system of claim 2, wherein the organic solvents are present in the range of from about 40 to about 60 wt % of the base coat nail polish composition; the film-forming compounds are present in the range of from about 20 to about 30 wt % of the base coat nail polish composition; the plasticizers are present in the range of from about 5 to about 15 wt % of the base coat nail polish composition; the one or more additives are present in the range of from about 0.01 to about 5 wt % of the base coat nail polish composition; and the additional components are present in the range of from about 5 to about 10 wt % of the base coat nail polish composition.

5. The nail polish composition system of claim 1, wherein the mixture of cyanoacrylates and a methacrylate of the top coat nail polish composition includes Beta-methoxyethyl cyanoacrylate, C1-C10 linear or branched alkyl cyanoacrylate, and polymethyl methacrylate.

6. The nail polish composition system of claim 5, wherein the Beta-methoxyethyl cyanoacrylate is present in the range of from about 70 to about 95 wt % of the mixture of cyanoacrylates and a methacrylate; the C1-C10 linear or branched alkyl cyanoacrylate is present in the range of from about 5 to about 20 wt % of the mixture of cyanoacrylates and a methacrylate; and the polymethyl methacrylate is present in the range of from about 1 to about 10 wt % of the mixture of cyanoacrylates and a methacrylate.

7. Atop coat nail polish composition for use in combination with a base coat nail polish composition, comprising a mixture of cyanoacrylates and a methacrylate.

8. The top coat nail polish composition of claim 7, the nail polish composition of claim 1, wherein the mixture of cyanoacrylates and a methacrylate of the top coat nail polish composition includes Beta-methoxyethyl cyanoacrylate, C1-C10 linear or branched alkyl cyanoacrylate, and polymethyl methacrylate.

9. The top coat nail polish composition of claim 8, wherein the Beta-methoxyethyl cyanoacrylate is present in the range of from about 70 to about 95 wt % of the mixture of cyanoacrylates and a methacrylate; the C1-C10 linear or branched alkyl cyanoacrylate is present in the range of from about 5 to about 20 wt % of the mixture of cyanoacrylates and a methacrylate; and the polymethyl methacrylate is present in the range of from about 1 to about 10 wt % of the mixture of cyanoacrylates and a methacrylate.

10. In combination, a base coat nail polish composition; and a top coat nail polish composition including a mixture of cyanoacrylates and a methacrylate.

11. The combination of claim 10, wherein the base coat nail polish composition includes organic solvents, film-forming compounds, plasticizers, and one or more additives.

12. The combination of claim 11, wherein the base coat nail polish composition includes additional components selected from a group consisting of pigments, anti-foaming agents, thickening agents, suspension agents, UV absorbers and/or a combination thereof.

13. The combination of claim 11, wherein the one or more additives include dimethyltolylamine.

14. The combination of claim 10, wherein the base coat nail polish composition incudes dimethyltolylamine.

15. The combination of claim 11, wherein the organic solvents are present in the range of from about 40 to about 60 wt % of the base coat nail polish composition; the film-forming compounds are present in the range of from about 20 to about 30 wt % of the base coat nail polish composition; the plasticizers are present in the range of from about 5 to about 15 wt % of the base coat nail polish composition; the one or more additives are present in the range of from about 0.01 to about 5 wt % of the base coat nail polish composition; and the additional components are present in the range of from about 5 to about 10 wt % of the base coat nail polish composition.

16. The combination of claim 10, wherein the mixture of cyanoacrylates and a methacrylate of the top coat nail polish composition includes Beta-methoxyethyl cyanoacrylate, C1-C10 linear or branched alkyl cyanoacrylate, and polymethyl methacrylate.

17. The combination of claim 16, wherein the Beta-methoxyethyl cyanoacrylate is present in the range of from about 70 to about 95 wt % of the mixture of cyanoacrylates and a methacrylate; the C1-C10 linear or branched alkyl cyanoacrylate is present in the range of from about 5 to about 20 wt % of the mixture of cyanoacrylates and a methacrylate; and the polymethyl methacrylate is present in the range of from about 1 to about 10 wt % of the mixture of cyanoacrylates and a methacrylate.

18. A method of applying a system of nail polish composition to a finger nail, comprising the steps of:
(a) applying a base coat nail polish composition, comprising organic solvents, film-forming compounds, plasticizers, and one or more additives, to the surface of the finger nail to form a base coat film;
(b) applying a top coat nail polish composition, comprising a mixture of cyanoacrylates and a methacrylate, over the first base coat film to form a top coat film.

19. The method of claim 18, wherein each of step (a) and step (b) is repeated once.

20. The method of claim 18, wherein each of step (a) and step (b) is repeated twice.

21. The method of claim 18, wherein the base coat nail polish composition further includes additional components selected from a group consisting of pigments, anti-foaming agents, thickening agents, suspension agents, UV absorbers and/or a combination thereof.

22. The method of claim 18, wherein the one or more additives are dimethyltolylamine.

23. The method of claim 18, wherein the organic solvents are present in the range of from about 40 to about 60 wt % of the base coat nail polish composition; the film-forming compounds are present in the range of from about 20 to about 30 wt % of the base coat nail polish composition; the plasticizers are present in the range of from about 5 to about 15 wt % of the base coat nail polish composition; the one or more additives are present in the range of from about 0.01 to about 5 wt % of the base coat nail polish composition; and the additional components are present in the range of from about 5 to about 10 wt % of the base coat nail polish composition.

24. The method of claim 18, wherein the mixture of cyanoacrylates and a methacrylate of the top coat nail polish composition comprising Beta-methoxyethyl cyanoacrylate, C1-C10 linear or branched alkyl cyanoacrylate, and polymethyl methacrylate.

25. The method of claim 24, wherein the Beta-methoxyethyl cyanoacrylate is present in the range of from about 70 to about 95 wt % of the mixture of cyanoacrylates and a methacrylate; the C1-C10 linear or branched alkyl cyanoacrylate is present in the range of from about 5 to about 20 wt % of the mixture of cyanoacrylates and a methacrylate; and the polymethyl methacrylate is present in the range of from about 1 to about 10 wt % of the mixture of cyanoacrylates and a methacrylate.
